(19) United States
(12) Patent Application Publication
(10) Pub. No.: US 2006/0286182 A1
(43) Pub. Date: Dec. 21, 2006

(54) Synergistic cinnamon combinations and methods for enhancing insulin activity

(75) Inventor: Dinesh C. Patel, Salt Lake City, UT (US)

Correspondence Address: THORPE NORTH & WESTERN, LLP., 8180 SOUTH 700 EAST, SUITE 200, SANDY, UT, 84070, US

(73) Assignee: Abeille Pharmaceuticals, Inc.

(21) Appl. No.: 11/158,289

(22) Filed: Jun. 21, 2005

(51) Int. Cl. A61K 36/54; A61K 31/426; A61K 31/175; A61K 31/155

(52) U.S. Cl. 424/739; 514/369; 514/592; 514/635

(57) ABSTRACT

Novel compositions and methods are provided for modifying adipocyte physiology in an animal subject such as a human. The methods include administering to the animal subject a novel pharmaceutical compositions derived from Cinnamomi cassia extracts and hypoglycemic therapeutics. Also provided are methods of increasing insulin sensitivity in an animal, which comprise administering to the animal an amount of a combination of cinnamon and metformin or glipizide sufficient to increase insulin sensitivity. Also provided are methods of treating disorders related to insulin resistance.

### FIELD OF THE INVENTION

The present invention relates to pharmaceutical compositions derived from Cinnamomi cassia extracts and hypoglycemic therapeutics that are useful for prevention and treatment of metabolic disorders, in particular, insulin resistance syndromes, type 2 diabetes, obesity, weight gain, and cardiovascular disease. More specifically, this invention relates to pharmaceutical compositions and therapeutic methods utilizing such compositions to modify adipocyte physiology to enhance lipogenesis.

### BACKGROUND OF THE INVENTION

Type 2 diabetes is by far the most common form of diabetes, accounting for 80-90% of diagnosed patients [Stumvoll, M. Control of glycaemia: from molecules to men. Minkowski Lecture 2003. Diabetologia, 47: 770-781, 2004]. Tissue insulin resistance and impaired insulin secretion characterize the underlying pathology of this disease. Until recently, only the pancreas, liver and muscle were considered essential to the understanding of the molecular dysregulation involved in the metabolic sequelae of insulin insensitivity, metabolic syndrome and type 2 diabetes.

However, it is now generally accepted that adipose tissue acts as an endocrine organ producing a number of biologically active peptides with an important role in the regulation of food intake, energy expenditure and a series of metabolic processes. Adipose tissue secretes a number of bioactive peptides collectively termed adipokines. Through their secretory function, adipocytes lie at the heart of a complex network capable of influencing several physiological processes. Dysregulation of adipokine production with alteration of adipocyte mass has been implicated in metabolic and cardiovascular complications of obesity. In obese individuals, excessive production of acylation-stimulating protein (ASP), tumor necrosis factor alpha (TNF□), interleukin-6 (IL-6) or resistin deteriorates insulin action in muscles and liver, while increased angiotensinogen and PAI-1 secretion favors hypertension and impaired fibrinolysis. Leptin regulates energy balance and exerts an insulin-sensitizing effect. These beneficial effects are reduced in obesity due to leptin resistance. Adiponectin increases insulin action in muscles and liver and exerts an anti-atherogenic effect. Further, adiponectin is the only known adipokine whose circulating levels are decreased in the obese state.

Insulin resistance and/or hyperinsulinemia have been postulated to be the cause of the other abnormal metabolic and cardiovascular risk factors that occur in the metabolic syndrome. These risk factors have been identified as (1) central obesity including increased visceral fat; (2) a characteristic dyslipidemia that includes an elevated plasma triglyceride, a low plasma high-density density lipoprotein (HDL), and a small dense low-density lipoprotein (LDL) cholesterol particle pattern; (3) a procoagulant state made up of elevated plasma fibrinogen and plasminogen activator inhibitor-1; (4) elevated systolic and diastolic blood pressure; (5) hyperuricemia; and (6) microalbuminuria [Lebovitz H E, Banerji M A. Insulin resistance and its treatment by thiazolidinediones. Recent Prog Horm Res. (2001) 56:265-94].

The biguanides, chloroguanide, phenformin, buformin and metformin, and the sulphonylureas, tolbutamide, chlorpropamide, tolazamide, acetohexamide, glyburide, gliclazide and glipizide, represent the two most commonly prescribed oral treatment options for type 2 diabetes. These two classes of agents have different mechanisms of action; biguanides, like metformin, act to improve insulin sensitivity and suppress hepatic glucose output, whereas sulphonylureas, such as glipizide, reduces hyperglycemia by enhancing insulin secretion. Molecular targets of these agents have recently been revealed; AMP-activated protein kinase (AMPK) for biguanides and dephosphorylation of key metabolic proteins/enzymes, like GLUT4 for sulfonylureas. Additionally, both therapeutic classes have been shown to improve glucose utilization in the adipocyte [Klein, J., Westphal, S., Kraus, D., Meier, B., Perwitz, N., Ott, V., Fasshauer, M., and Klein, H. H. Metformin inhibits leptin secretion via a mitogen-activated protein kinase signalling pathway in brown adipocytes. J Endocrinol, 183: 299-307, (2004); Pedersen, O., Nielsen, O., Bak, J., Richelsen, B., Beck-Nielsen, H., and Sorensen, N. The effects of metformin on adipocyte insulin action and metabolic control in obese subjects with type 2 diabetes. Diabet Med, 6: 249-256, (1989); Pryor, P. R., Liu, S. C., Clark, A. E., Yang, J., Holman, G. D., and Tosh, D. Chronic insulin effects on insulin signalling and GLUT4 endocytosis are reversed by metformin. Biochem J, 348 Pt 1: 83-91, (2000); Stolar, M. W. Insulin resistance, diabetes, and the adipocyte. Am J Health Syst Pharm, 59 Suppl 9. S3-8, (2002)].

Thiazoledineiones (TZDs) are a third group of compounds used in the treatment of type 2 diabetes since 1997. These compounds represent the newest class of oral anti-diabetic drugs and are often referred to as insulin sensitizers. TZDs function as ligands for the peroxisome proliferator-activated receptor gamma (PPARγ) located in the nucleus of adipocytes. They are characterized by their ability to decrease insulin resistance, and have been suggested to slow down the progression of insulin resistance. The first of this class was ciglitazone, which was synthesized in 1982, followed by pioglitazone, englitazone, troglitazone, rosiglitazone and darglitazone. Only troglitazone, pioglitazone and rosiglitazone were evaluated in clinical studies. Troglitazone, approved for use in the US in 1997, was withdrawn from the market early in 2000 because of idiosyncratic liver toxicity [Larsen T M, Toubro S, Astrup A. PPARgamma agonists in the treatment of type II diabetes: is increased fatness commensurate with long-term efficacy? Int J Obes Relat Metab Disord. February 2003; 27(2):147-161]. Rosiglitazone (Avandia from GlaxoSmithKline) and pioglitazone (Actos from Eli Lily) were approved by the US Food and Drug Administration in 1999 and currently are used by approximately three million type 2 diabetics in the US [Schmitz. O E, Brock B, Madsbad S, Beck-Nielsen H. [Thiazolidinediones—a new class of oral antidiabetics]. Ugeskr Laeger. Oct. 29 2001; 163(44):6106-6111].

Rosiglitazone, pioglitazone and troglitazone display unique pharmacological characteristics, although they all contain the same active TZD ring. The distinct pharmacological activities among these synthetic PPARY ligands are believed to be due to the dissimilarity in side chains. As a result, these PPARγagonists differ in their pharmacological potency; the PPARγ-binding affinity of rosiglitazone is 100-fold greater than that of troglitazone and over 30 times that of pioglitazone [Young P W, Buckle D R, Cantello B C, et al. Identification of high-affinity binding sites for the insulin sensitizer rosiglitazone (BRL-49653) in rodent and human adipocytes using a radioiodinated ligand for peroxisomal proliferator-activated receptor gamma. J Pharmacol Exp Ther. February 1998; 284(2):751-759]. The rank order of binding affinities of the PPARγ-agonists, rosiglitazone>pioglitazone>troglitazone, is consistent with their dose requirements for in vitro stimulation of glucose transport and their anti-hyperglycemic activity in ob/ob mice [Young P W, Buckle D R, Cantello B C, et al. Identification of high-affinity binding sites for the insulin sensitizer rosiglitazone (BRL-49653) in rodent and human adipocytes using a radioiodinated ligand for peroxisomal proliferator-activated receptor gamma. J Pharmacol Exp Ther. February 1998; 284(2):751-759]. Their respective effectiveness likely reflect their ability to induce adipocyte differentiation, and hence to increase free fatty acid uptake in white adipose tissue.

Despite their different degrees of potency, all PPARγagonists have been shown to be effective in relieving insulin resistance. The beneficial metabolic effects of PPARγagonist treatment of type 2 diabetes include: (1) reduction in postprandial glucose, fasting plasma glucose and gylcosylated hemoglobin; (2) increased insulin sensitivity and improved pancreatic island β-cell function; (3) increased HDL levels and variable lowering of LDL levels; (4) lowering of diastolic blood pressure, decreased microalbuminurea, and increased levels of the fibrinolytic plasminogen activator inhibitor 1 (PAI-1) and tissue plasminogen activator (tPA) [Zinman B. PPAR gamma agonists in type 2 diabetes: how far have we come in ‘preventing the inevitable’? A review of the metabolic effects of rosiglitazone. Diabetes Obes Metab. August 2001; 3 Suppl 1:S34-43].

While the TZDs have demonstrated good efficacy for increasing insulin sensitivity as well as a range of additional beneficial effects, many patients do not achieve a large enough hypoglycemimic response to eliminate the use of insulin or other insulinotropic drugs, and many patients are completely nonresponsive to this class of drugs. Additionally, adverse effects seen with TZDs include weight gain, edema, upper respiratory tract infection and headache [Larsen T M, Toubro S, Astrup A. PPARgamma agonists in the treatment of type II diabetes: is increased fatness commensurate with long-term efficacy? Int J Obes Relat Metab Disord. February 2003; 27(2):147-161]. Thus, there is a need for combinations of dietary supplements, foods or drugs that will increase the percentage of patients responding to TZDs and/or decrease the side effects of weight gain, edema, upper respiratory tract infection and headache associated with this class of drugs.

Based on available evidence, metformin monotherapy is preferred for the vast majority of type 2 diabetic patients who are overweight or obese [Lebovitz, H. E. and Banerji, M. A. Treatment of insulin resistance in diabetes mellitus. Eur J Pharmacol, 490: 135-146, (2004)]. Combination therapy has further improved glycemic control. However, limitations in use, including the challenges of side effects, to that of secondary oral agent failure will inevitably occur over time [Gin, H. and Rigalleau, V. Oral anti diabetic polychemotherapy in type 2 diabetes mellitus. Diabetes Metab, 28: 350-353, (2002); McCarty, M. F. Complementary measures for promoting insulin sensitivity in skeletal muscle. Med Hypotheses, 51: 451-464, (1998)].

These challenges leave ample room for the development of agents that address the pathophysiology not only of treating insulin resistance and decreasing insulin production but also of preventing or delaying the development of diabetes in populations at risk. Evidence has been published that a wide array of plant-derived active principles, representing numerous classes of chemical compounds, demonstrate activity consistent with their possible use in the treatment of patients with type 2 diabetes mellitus [Preuss, H. G., Bagchi, D., and Bagchi, M. Protective effects of a novel niacin-bound chromium complex and a grape seed proanthocyanidin extract on advancing age and various aspects of syndrome X. Ann N Y Acad Sci, 957: 250-259, (2002); Al-Awwadi, N., Azay, J., Poucheret, P., Cassanas, G., Krosniak, M., Auger, C., Gasc, F., Rouanet, J. M., Cros, G., and Teissedre, P. L. Antidiabetic activity of red wine polyphenolic extract, ethanol, or both in streptozotocin-treated rats. J Agric Food Chem, 52: 1008-1016, (2004); Virgili, F., Kobuchi, H., and Packer, L. Procyanidins extracted from Pinus maritima (Pycnogenol): scavengers of free radical species and modulators of nitrogen monoxide metabolism in activated murine RAW 264.7 macrophages. Free Radic Biol Med, 24: 1120-1129, (1998); Sevin, R. and Cuendet, J. F. Effect of a combination of myrtillus anthocyanosides and beta-carotene on capillary resistance in diabetes]. Ophthalmologica, 152: 109-117, (1966)].

A study by Broadhurst et al. has demonstrated that cinnamon is a strong potentiator of insulin in comparison to various other herbs and spices [J. Agric. Food Chem., 2000; 48:849-852]. One particular cinnamon extract consisting of methyl-hydroxy-chalcone polymer (MHCP), showed promising results in the area of glucose control. A recent study compared the effect of MHCP in 3T3-L1 adipocytes to that of insulin. [Jarvill-Taylor et al., J. Am. College Nutr., 2001; 20:327-336]. The results from that study support the theory that MHCP triggers the insulin cascade and subsequent transport of nutrients. The study also demonstrated that MHCP treatment stimulated glucose uptake and glycogen synthesis to a similar level as insulin. The study further demonstrated that treatment with endogenous insulin and MHCP resulted in synergistic effects.

Due to these conclusions it is suggested that MHCP may prove to be a very valuable tool in the fight against diabetes where insulin is present. In addition to benefiting type 2 diabetics, they may benefit individuals with impaired glucose tolerance (i.e., pre-diabetics). Further, MHCP has been shown to possess antioxidant activities related to lipid peroxidation. [Mancini-Filho et al., Bollettino ChimicoFarmaceutico, 1998; 37:443-47] and can be used as a food antioxidant and to enhance food palatability.

Despite these interesting observations, to date, metformin is the only ethical drug approved for treatment of type 2 diabetes derived from a medicinal plant. Thus, there is a need for assessing combinations of drugs and natural products that may extend the clinical usefulness of the drug. However, to date no research has demonstrated synergy of cinnamon or cinnamon extracts with current oral therapies for hyperglycemia. It was, therefore, the objective of these studies to assess the lipogenic effect and potential synergy of metformin, glipizide and pioglitazone in combination with ground cinnamon and a cinnamon extract in the 3T3-L1 adipocyte model.

Despite advances in treating type 2 diabetes in recent years, there remains a need for compositions for treatment and prevention of diabetes and diabetes-related conditions and disorders, such as insulin resistance and metabolic syndrome X. With the aforementioned increase in the incidence of obesity, compositions and methods for treatment and prevention of obesity are also needed. The present invention satisfies these needs and provides related advantages as well.

### SUMMARY OF THE INVENTION

Disclosed herein is: (a) a pharmaceutical composition comprising cinnamon powder or an extract thereof or a derivative of the extract thereof and a hypoglycemic therapeutic selected from the group consisting of a biguanide, a sulfonylurea, a thiazolidinedione and mixtures thereof; and (b) methods of using the composition thereof to modify adipocyte physiology in a subject. Preferably, the hypoglycemic therapeutic is member selected from the group consisting of metformin, glipizide and pioglitazone, or a derivative or a precursor thereof. The present invention relates to the unexpected discovery that combinations of cinnamon powder or an extract thereof and a hypoglycemic therapeutic increased adipocyte lipogenesis more effectively than the individual components or the expected additive effect of the individual components. Preferred embodiments provide compositions and methods for enhancing adipocyte lipogenesis

In some embodiments, the invention provides a method of increasing insulin sensitivity employing a composition comprising cinnamon powder or an extract thereof or a derivative of the extract thereof and at least one member of the group consisting of metformin, glipizide or pioglitazone as described in more detail herein.

The present invention further provides a composition of matter, comprising cinnamon powder or an extract thereof or a derivative of the extract thereof and at least one member of the group consisting of metformin, glipizide and pioglitazone. It has been surprisingly found that complex combinations of these compounds result in a greater increase of adipocyte lipogenesis than individual compounds. Preferred embodiments provide compositions and methods for enhancing adipocyte lipogenesis.

Thus, combinations of cinnamon powder or an extract thereof or a derivative of the extract thereof and at least one member of the group consisting of metformin, glipizide or pioglitazone possess superior activity for the treatment and prevention of an number of metabolic and inflammatory conditions, including: type 2 diabetes mellitus, syndrome X, diabetic complications, hyperlipidemia, obesity, osteoporosis, inflammatory diseases, diseases of the digestive organs, stenocardia, myocardial infarction, sequelae of stenocardia or myocardial infarction, senile dementia, and cerebrovascular dementia. see, Harrison's Principles of Internal Medicine, 13th Ed., McGraw Hill Companies Inc., New York (1994).

In other embodiments, the invention provides a composition, comprising at least two pharmaceutically active agents, wherein the pharmaceutical active agents comprise a cinnamon powder or a cinnamon extract and at least one member of the group consisting of biguanides, sulfonylureas and thiazolidinediones.

The invention further provides a method of increasing insulin sensitivity in a subject, comprising administering to the subject an insulin sensitivity increasing amount of a cinnamon powder or a cinnamon extract and at least one member of the group consisting of biguanides, sulfonylureas, and thiazolidinediones from whatever source derived, including a salt, such as a pharmaceutically acceptable salt, tautomer or isomer thereof.

The invention further provides methods for the treatment of diabetes mellitus, hyperglycemia, syndrome X, type 2 diabetes, diabetic complications, hyperlipidemia, obesity, osteoporosis, inflammatory disease, a disease of the digestive organs, stenocardia, myocardial infarction, sequelae of stenocardia or myocardial infarction, senile dementia, or cerebrovascular dementia. The methods include administering to a subject an effective amount of a composition of the invention comprising cinnamon powder or a cinnamon extract and at least one member of the group consisting of biguanides, sulfonylureas, and thiazolidinediones, including salts, such as pharmaceutically acceptable salts, tautomers and isomers thereof.

### BRIEF DESCRIPTION OF THE FIGURES

FIG. 1 represents a schematic of the testing protocol;

FIG. 2 is a bar graph representing the relative triglyceride content of 3T3-L1 adipocytes following treatment of indicated test materials and combinations;

FIG. 3 is a bar graph representing the relative triglyceride content of 3T3-L1 adipocytes following treatment of indicated test materials and combinations;

FIG. 4 represents the relative triglyceride content of 3T3-L1 adipocytes following treatment of indicated test materials and combinations;

FIG. 5 represents the relative triglyceride content of 3T3-L1 adipocytes following treatment of indicated test materials and combinations.

### DETAILED DESCRIPTION OF THE INVENTION

Before the present invention is disclosed and described, it is to be understood that this invention is not limited to the particular structures, process steps, or materials disclosed herein, but is extended to equivalents thereof as would be recognized by those ordinarily skilled in the relevant arts. It should also be understood that terminology employed herein is used for the purpose of describing particular embodiments only and is not intended to be limiting.

Definitions

In describing and claiming the present invention, the following terminology will be used.

The singular forms “a,” “an,” and “the” include plural referents unless the context clearly dictates otherwise. The term “formulation” and “composition” may be used interchangeably herein.

The phrases “therapeutically effective amount” refers to an amount of the formulation consisting of ground cinnamon or a cinnamon extract and a hypoglycemic therapeutic selected from the group consisting of a biguanide, a sulfonylurea, a thiazolidinedione and mixtures thereof sufficient to achieve therapeutic results as desired. In the formulation, the active ingredients are present in association with a pharmaceutically acceptable vehicle and optionally one or more other therapeutic ingredients. The vehicle must be “pharmaceutically acceptable” in the sense of being compatible with the other ingredients of the formulations and not deleterious to the recipient thereof. The formulation may be in a form suitable for oral, ophthalmic, rectal, parenteral (including subcutaneous, intramuscular, interperitoneal, intraarticular and intravenous), transdermal, and topical, nasal or buccal administration.

The formulations may conveniently be presented in dosage unit form and may be prepared by any of the methods well known in the art of pharmacy. All methods include the step of bringing the active ingredient into association with the vehicle which constitutes one or more auxiliary constituents, In general, the formulations are prepared by uniformly and intimately bringing the active ingredient into association with a liquid vehicle or a finely divided solid vehicle or both, and then, if necessary, shaping the product into the desired formulation.

The term “dosage unit” is understood to mean a unitary, i.e. a single dose which is capable of being administered to a patient, and which may be readily handled and packed, remaining as a physically and chemically stable unit dose comprising either the active ingredient as such or a mixture of it with solid or liquid pharmaceutical vehicle materials.

Formulations suitable for oral administration may be in the form of discrete units as capsules, sachets, tablets, beads or lozenges, each containing a predetermined amount of the active ingredient; in the form of a powder or granules; in the form of a solution or a suspension in an aqueous liquid or non-aqueous liquid, such as ethanol or glycerol; or in the form of an oil-in-water emulsion or a water-in-oil emulsion. Such oils may be edible oils, such as e.g. cottonseed oil, sesame oil, coconut oil or peanut oil. Suitable dispersing or suspending agents for aqueous suspensions include synthetic or natural gums such as tragacanth, alginate, acacia, dextran, sodium carboxymethylcellulose, gelatin, methylcellulose and polyvinylpyrrolidone. The active ingredient may also be administered in the form of a bolus, electuary or paste. Transdermal formulations may be in the form of a plaster.

Formulations suitable for ophthalmic administration may be in the form of a sterile aqueous preparation of the active ingredients, which may be in microcrystalline form, for example, in the form of an aqueous microcrystalline suspension. Liposomal formulations or biodegradable polymer systems may also be used to present the active ingredient for ophthalmic administration.

Formulations suitable for topical or ophthalmic administration include liquid or semi-liquid preparations such as liniments, lotions, gels, oil-in-water or water-in-oil emulsions such as creams, ointments or pastes; or solutions or suspensions such as drops.

In addition to the aforementioned ingredients, the formulations comprising Cinnamomi cassia or its derivatives of the present invention may also comprises one or more additional ingredients such as diluents, buffers, flavoring agents, colorants, surface active agents, thickeners, preservatives, e.g. methyl hydroxybenzoate (including anti-oxidants), emulsifying agents and the like.

In addition to the formulations described above, Cinnamomi cassia powders or extracts may also be formulated as a depot preparation. Such long-acting formulations may be administered by implantation (e.g. subcutaneously or intramuscularly) or by intramuscular injection. Thus, for example, the active ingredient may be formulated with suitable polymeric or hydrophobic materials (for example as an emulsion in a pharmaceutically acceptable oil), or an ion exchange resin.

For systemic treatment according to the present invention, daily doses of from 0.001-200 mg/kg body weight, preferably from 0.002-20 mg/kg of mammal body weight, for example 0.003-10 mg/kg of the composition of the present invention are administered, corresponding to a daily dose for an adult human of from 0.2 to 14000 mg of the active ingredient. In the topical treatment of dermatological disorders, ointments, creams or lotions containing from 0.1-750 mg/g, and preferably from 0.1-500 mg/g, of the composition of the present invention may be administered. For topical use in ophthalmological ointments, drops or gels containing from 0.1-750 mg/g, and preferably from 0.1-500 mg/g, of the composition of the present invention are administered. Oral compositions are formulated, preferably as tablets, capsules, or drops, containing from 0.05-250 mg, preferably from 0.1-1000 mg, of the composition of the present invention per dosage unit.

The composition of this invention can be administered in a convenient formulation. The following formulation examples only are illustrative and are not intended to limit the scope of the present invention. In the formulations that follow, “active ingredient” means a composition of this invention.

Formulation 1: Gelatin Capsules—Hard gelatin capsules are prepared using the following ingredient quantity (mg/capsule) (1) Active ingredient 0.15-1000 (2) Starch, NF 0-650 (3) Starch flowable powder 0-50 (4) Silicone fluid 350 centistokes 0-15.

A tablet formulation is prepared using the ingredients below:

Formulation 2: Tablets—Ingredient quantity (mg/tablet)—(1) Active ingredient 0.25-500 Cellulose, microcrystalline 200-650, Silicon dioxide, fumed 10-650, stearic acid 5-15 The components are blended and compressed to form tablets.

Alternatively, tablets each containing 0.25-500 mg of active ingredients are made up as follows:

Formulation 3: Tablets Ingredient Quantity (mg/tablet)—(1) Active ingredient 0.25-500, (2) Starch 45 Cellulose, (3) microcrystalline 35 Polyvinylpyrrolidone (as 10% solution in water,) (4) Sodium carboxymethyl cellulose 4.5 (5) Magnesium stearate 0.5 (6) Talc 1 The active ingredients, starch, and cellulose are passed through a No. 45 mesh U.S. sieve and mixed thoroughly. The solution of polyvinylpyrrolidone is mixed with the resultant powders that are then passed through a No. 14 mesh U.S. sieve. The granules so produced are dried at 50-60° C. and passed through a No. 18 mesh U.S. sieve. The sodium carboxymethyl starch, magnesium stearate, and talc, previously passed through a No. 60 U.S. sieve, are then added to the granules that, after mixing, are compressed on a tablet machine to yield tablets.

Suspensions each containing 0.25-500 mg of active ingredient per 5 ml dose are made as follows:

Formulation 4: Suspensions Ingredient Quantity (mg/5 ml)—(1) Active ingredient 0.25-500 mg, (2) Sodium carboxymethyl cellulose 50 mg (3) Syrup 1.25 mg Benzoic acid solution 0.10 mL (4) Flavor q.v. Color q.v. (5) Purified Water to 5 mL

The active ingredient is passed through a No. 45 mesh U.S. sieve and mixed with the sodium carboxymethyl cellulose and syrup to form a smooth paste. The benzoic acid solution, flavor, and color are diluted with some of the water and added, with stirring. Sufficient water is then added to produce the required volume.

An aerosol solution is prepared containing the following ingredients:

Formulation 5: Aerosol Ingredient Quantity (% by weight)—(1) Active ingredient 0.25, (2) ethanol 25.75, (3) Propellant 22 (chlorodifluoromethane) 70.00. The active ingredient is mixed with ethanol and the mixture added to a portion of the propellant 22, cooled to 30° C., and transferred to a filling device. The required amount is then fed to a stainless steel container and diluted with the remaining propellant. The valve units are then fitted to the container.

Suppositories are prepared as follows:

Formulation 6: Suppositories—Ingredient Quantity (mg/suppository)—(1) Active ingredient 250, (2) Saturated fatty acid glycerides 2,000.

The active ingredient is passed through a No. 60 mesh U.S. sieve and suspended in the saturated fatty acid glycerides previously melted using the minimal necessary heat. The mixture is then poured into a suppository mold of nominal 2 g capacity and allowed to cool.

An intravenous formulation is prepared as follows:

Formulation 7: Intravenous Solution—Ingredient Quantity—(1) active ingredient dissolved in ethanol 1% (2) 20 mg Intralipid™ emulsion 1,000 mL.

The solution of the above ingredients is intravenously administered to a patient at a rate of about 1 mL per minute.

The active ingredient above may also be a combination of hypoglycemic, therapeutic agents. The ingredients can be administered in a single formulation or they can be separately administered. Thus, the invention also provides a pharmaceutical pack or kit comprising one or more containers filled with one or more of the ingredients of the compositions of the invention (e.g., capsules or pills containing a cinnamon powder or extract and at least one member of the group consisting of a sulfonylurea, biguanide and thiazolidinedione). Optionally associated with such container(s) can be a notice in the form prescribed by a government agency regulating the manufacture, use or sale of pharmaceutical products, which notice reflects approval by the agency of manufacture, use of sale for human administration. The pack or kit can be labeled with information regarding mode of administration, sequence of administration (e.g., separately, sequentially or concurrently), or the like. The pack or kit may also include means for reminding the patient to take the therapy. The pack or kit can be a single unit dosage of the combination therapy or it can be a plurality of unit dosages. In particular, the agents can be separated, mixed together in any combination, present in a formulation or tablet.

Concentrations, amounts, solubilities, and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges encompassed within that range as if each numerical value and sub-range is explicitly recited. As an illustration, a concentration range of “about 1% w/w to about 10% w/w” should be interpreted to include not only the explicitly recited concentration of about 1% to about 10% w/w, but also include individual concentrations and the sub-ranges within the indicated range. Thus, included in this numerical range are individual concentrations such as 2% w/w, 5% w/w, and 8% w/w, and sub-ranges such as from 1% w/w to 3% w/w, from 2% w/w to 4% w/w, from 3% w/w to 8% w/w, from 5% w/w to 9% w/w, from 1% w/w to 7% w/w etc. The same principle applies to ranges reciting only one numerical value.

As used herein, the term “subject” refers to an animal, particularly a mammal, or a human, to which a composition is administered. The methods of the present invention are intended for use with any subject that may experience the benefits of the methods of the invention. Thus, in accordance with the invention, “subject” includes both those who are being treated for a particular malady or disorder, commonly referred to as a patient, and those to whom the composition is administered in order to elicit a prophylactic or preventative response.

As used herein, the term “hyperlipidemia” refers to a pathognomic condition manifest by elevated serum concentrations of total cholesterol (>200 mg/dL), LDL cholesterol (>130 mg/dL), or triglycerides (>150 mg/dL) or decreased HDL cholesterol (<40 mg/dL) or other appropriate markers such as C-reactive protein, Tumor Necrosis Factor alpha (TNFα), Interleukin-6 (IL-6), adiponectin, leptin or resistin. Further, as used herein, the term “fat” refers to serum and adipose triglyceride content and “triglycerides” refers to triacylglyerol esters of fatty acids. As used herein “fat cell” refers to the adipocyte, the major constituent of white adipose tissue in the body.

As used herein, the terms “hyperinsulinemia” and “hyperglycemia” refer, respectively, to a fasting insulin concentration>17 IU/mL) and fasting glucose>125 mg/dL.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

As used in this specification, whether in a transitional phrase or in the body of the claim, the terms “comprise(s)” and “comprising” are to be interpreted as having an open-ended meaning. That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

As used herein, the term “insulin sensitivity” refers to the ability of a cell, tissue, organ or whole body to absorb glucose in response to insulin. As used in an in vivo sense, “insulin sensitivity” refers to the ability of an organism to absorb glucose from the blood stream. An improvement in insulin sensitivity therefore results in an improved ability of the organism to maintain blood glucose levels within a target range. Thus, improved insulin sensitivity also results in decreased incidence of hyperglycemia. Improved insulin sensitivity can also treat, prevent or delay the onset of various metabolic conditions, such as diabetes mellitus, syndrome X and diabetic complications. Because of the improved metabolic processing of dietary sugar, improved insulin sensitivity can also treat, prevent or delay the onset of hyperlipidemia and obesity. Additionally, improved insulin sensitivity can lead to treatment, prevention or delayed onset of a variety of inflammatory conditions, such as diseases of the digestive organs (such as ulcerative colitis, Crohn's disease, pancreatitis, gastritis, benign tumor of the digestive organs, digestive polyps, hereditary polyposis syndrome, colon cancer, rectal cancer, stomach cancer and ulcerous diseases of the digestive organs), stenocardia, myocardial infarction, sequelae of stenocardia or myocardial infarction, senile dementia, cerebrovascular dementia, immunological diseases and cancer in general.

As used herein “diabetic complications” include retinopathy, muscle infarction, idiopathic skeletal hyperostosis and bone loss, foot ulcers, neuropathy, arteriosclerosis, respiratory autonomic neuropathy and structural derangement of the thorax and lung parenchyma, left ventricular hypertrophy, cardiovascular morbidity, progressive loss of kidney function, and anemia.

As used herein, the term “effective amount” means, depending on the context, an amount sufficient to treat, prevent or delay the onset of a particular condition.

The term “treat” and its verbal variants refer to palliation or amelioration of an undesirable physiological state. Thus, for example, where the physiological state is poor glucose tolerance, “treatment” refers to improving the glucose tolerance of a treated subject. As another example, where the physiological state is obesity, the term “treatment” refers to reducing the body fat mass, improving the body mass or improving the body fat ratio of a subject. Treatment of diabetes means improvement of blood glucose control. Treatment of inflammatory diseases means reducing the inflammatory response either systemically or locally within the body. Treatment of cancer means reduction in hyperproliferation, inducement of cell death in cancer cells or reduction in metastasis. The person skilled in the art will recognize that treatment may, but need not always, include remission or cure.

The term “prevent” and its variants refer to prophylaxis against a particular undesirable physiological condition. The prophylaxis may be partial or complete. Partial prophylaxis may result in the delayed onset of a physiological condition. The person skilled in the art will recognize the desirability of delaying onset of a physiological condition, and will know to administer the compositions of the invention to subjects who are at risk for certain physiological conditions in order to delay the onset of those conditions. For example, the person skilled in the art will recognize that obese subjects are at elevated risk for coronary artery disease. Thus, the person skilled in the art will administer compositions of the invention in order to increase insulin sensitivity in an obese, whereby the onset of diabetes mellitus may be prevented entirely or delayed.

As used herein, the term “dietary supplement” refers to compositions consumed to affect structural or functional changes in physiology. The term “therapeutic composition” refers to any compounds administered to treat or prevent a disease.

As used herein, the terms “derivatives” or a matter “derived” refer to a chemical substance related structurally to another substance and theoretically obtainable from it, i.e. a substance that can be made from another substance. Derivatives can include compounds obtained via a chemical reaction.

As used herein, the terms “Cinnamomi cassia extract” and “cinnamon extract” refers to the solid material resulting from (1) exposing a Cinnamomi cassia product to a solvent, (2) separating the solvent from the Cinnamomi cassia plant products, and (3) eliminating the solvent.

As used herein, the terms “cinnamon powder” refers to a product obtained by grinding the bark of the Cinnamomi cassia.

When broadly used as, the terms “Cinnamon” the term is inclusive of both cinnamon extract and cinnamon powder.

As used herein, the term “hypoglycemic therapeutic” refers to a composition that functions systemically in an animal to normalize fasting insulin or glucose concentrations. Such formulations may also normalize serum cholesterol and triglycerides.

As used herein, the term “solvent” refers to a liquid of aqueous or organic nature possessing the necessary characteristics to extract solid material from the Cinnamomi cassia plant material. Examples of solvents would include, but not limited to, water, steam, superheated water, glycerin, ethylene glycol, methanol, diethylene glycol, ethanol, acetic acid, 1-propoanol, 1-butanol, acetonitrile, dimethyl sulfoxide, dimethyl formamide, t-butyl alcohol, acetone, 2-butanone, methylene chloride, chloroform, diglyme, dimethyoxy ethane, ethyl acetate, thetrahydrofuran, dioxane, methyl t-butyl ether, ether, benzene, toluene, p-xylene, carbon tetrachloride, heptane, hexane, pentane, octanol, cyclohexane, supercritical CO₂, liquid CO₂, liquid N₂ or any combinations of such materials. As used herein, the term “CO₂ extract” refers to the solid material resulting from exposing a Cinnamomi cassia plant product to a liquid or supercritical CO₂ preparation followed by removing the CO₂.

As used herein, “pharmaceutically acceptable carrier” includes solvents, dispersion media, coatings, isotonic and absorption delaying agents, sweeteners and the like.

The present invention relates to the unexpected discovery that a pharmaceutical composition comprising cinnamon powder or an extract thereof or a derivative of the extract thereof and a hypoglycemic therapeutic selected from the group consisting of a biguanide, a sulfonylurea, a thiazolidinedione and mixtures thereof produces a greater than expected increase in adipocyte lipogenesis than combinations of individual cinnamon extracts and hypoglycemic therapeutics. Preferred embodiments provide compositions and methods for enhancing adipocyte lipogenesis.

Preferred embodiments comprise compositions containing fractions or compounds isolated or derived from Cinnamomi cassia or cinnamon and at least one member selected from the group comprising biguanides, sulfonylureas and thiazolidinediones. Most preferred embodiments comprise compositions containing fractions or compounds isolated or derived from Cinnamomi cassia or cinnamon and at least one member selected from the group comprising metformin, glipizide and pioglitazone.

The invention provides a method of improving insulin sensitivity in a subject, comprising administering to the subject a composition comprising fractions or compounds isolated or derived from Cinnamomi cassia or cinnamon and at least one member selected from the group comprising biguanides, sulfonylureas and thiazolidinediones. The most preferred method of improving insulin sensitivity in a subject comprising administering to the subject a composition comprising fractions or compounds isolated or derived from Cinnamomi cassia or cinnamon and at least one member selected from the group comprising metformin, glipizide and pioglitazone.

In regard to improvement of insulin sensitivity, then, a subject may be an animal or human who has been diagnosed with insulin resistance or an animal or human, such as an obese or aged animal or human, which is determined to be at risk for insulin resistance. The ordinary clinician will be able to diagnose insulin resistance and, via analysis of a subject's health history, determine whether the subject is at risk for insulin resistance.

In general, an increase in adipocyte lipogenesis will result in improved insulin sensitivity resulting in improves glucose metabolism, improved blood lipid profiles, and decreased pro-inflammatory adipokine secretion. A decrease in pro-inflammatory adipokine secretion leads to decreased systemic inflammation and disorders associated with inflammation, such as diabetic complications, obesity, inflammatory diseases of the digestive organs, proliferative diseases of the digestive organs, ulcerous diseases of the digestive organs, stenocardia, myocardial infarction, sequelae of stenocardia, sequelae of myocardial infarction, senile dementia, cerebrovascular dementia, immunological diseases and cancer [Guerre-Millo, M. (2004) Adipose tissue and adipokines: for better or worse. Diabetes Metabolism 30:13-19].

The present invention also provides a composition of matter, comprising Cinnamomi cassia or cinnamon and at least one member selected from the group consisting of metformin, glipizide and pioglitazone. These combinations possess exceptional insulin sensitizing, anti-obesity and hypoglycemic activity.

When formulating combinations that have demonstrated synergy in vitro, it is necessary to remember that absorption, distribution and elimination will no doubt differ between the compounds in the formulation. As a result, serum or target tissue concentrations of the drugs will pass in and out of the ranges of synergy with each dose. Thus, the ratios of the drugs in the dosing formulations should not be limited to only the ranges of synergy demonstrated in vitro.

The present invention also provides a composition of matter, comprising Cinnamomi cassia or cinnamon and at least one member selected from the group consisting of metformin, glipizide and pioglitazone. These combinations possess exceptional insulin sensitizing, anti-obesity and anti-inflammatory activity.

Compositions of the invention are conveniently obtained from cinnamon (Cinnamomi cassia). Briefly, cinnamon may be extracted with a variety of solvents, such as water, steam, superheated water, glycerin, ethylene glycol, methanol, diethylene glycol, ethanol, acetic acid, 1-propoanol, 1-butanol, acetonitrile, dimethyl sulfoxide, dimethyl formamide, t-butyl alcohol, acetone, 2-butanone, methylene chloride, chloroform, diglyme, dimethyoxy ethane, ethyl acetate, thetrahydrofuran, dioxane, methyl t-butyl ether, ether, benzene, toluene, p-xylene, carbon tetrachloride, heptane, hexane, pentane, octanol, cyclohexane, supercritical CO₂, liquid CO₂, liquid N₂ or any combinations of such materials.

The invention provides a method of increasing insulin sensitivity in a subject, comprising administering to the subject an insulin sensitivity increasing amount of Cinnamomi cassia or cinnamon extract and at least one member selected from the group comprising biguanides, sulfonylureas, and thiazolidinediones from whatever source derived, including a salt, such as a pharmaceutically acceptable salt, tautomer or isomer thereof.

The invention further provides a method of increasing insulin sensitivity in a subject, comprising administering to the subject an insulin sensitivity increasing amount of Cinnamomi cassia or cinnamon extract and at least one member selected from the group comprising metformin, glipizide, and pioglitazone from whatever source derived, including a salt, such as a pharmaceutically acceptable salt, tautomer or isomer thereof.

The invention further provides methods for the treatment of diabetes mellitus, hyperglycemia, syndrome X, type 2 diabetes, diabetic complications, hyperlipidemia, obesity, osteoporosis, inflammatory disease, a disease of the digestive organs, stenocardia, myocardial infarction, sequelae from stenocardia or myocardial infarction, senile dementia, cerebrovascular dementia, an immunological disease or cancer. The methods include administering to a subject an effective amount of a composition of the invention comprising Cinnamomi cassia or cinnamon extract and at least one member selected from the group consisting of biguanides, sulfonylureas, and thiazolidinediones including salts, such as pharmaceutically acceptable salts, tautomers and isomers thereof.

Cinnamon and cinnamon extracts are available commercially for example from Viable Herbal Solutions (Morrisville, Pa.). Biguanides, sulfonylureas and thiazolidinediones are available commercially from, respectively, Bristol-Myers Squib (Princeton, N.J.), Aventis, (Parsippany, N.J.) and SmithKline Beecham (Philadelphia, Pa.).

In addition to two or more pharmaceutically active agents, the composition for dietary application may include various additives such as other natural components of intermediary metabolism, vitamins and minerals, as well as inert ingredients such as talc and magnesium stearate that are standard excipients in the manufacture of tablets and capsules.

Compositions of the invention include two or more pharmaceutically active agents in combination with one or more pharmaceutically acceptable carriers. These pharmaceutically acceptable carriers may be prepared from a wide range of materials including, but not limited to, diluents, binders and adhesives, lubricants, disintegrants, coloring agents, bulking agents, flavoring agents, sweetening agents and miscellaneous materials such as buffers and absorbents that may be needed in order to prepare a particular therapeutic composition. The use of such media and agents for pharmaceutically active substances is well known in the art. Except insofar as any conventional media or agent is incompatible with the active ingredients, its use in the present composition is contemplated. Other ingredients known to affect the manufacture of this composition as a dietary bar or functional food can include flavorings, sugars, amino-sugars, proteins and/or modified starches, as well as fats and oils.

Compositions of the invention may be in the form of dietary supplements, or therapeutic compositions. The dietary supplements, or therapeutic compositions of the present invention can be formulated in any manner known by one of skill in the art. In one embodiment, the composition is formulated into a capsule, tablet or bead using techniques available to one of skill in the art. In capsule, tablet or bead form, the recommended daily dose for an adult human or animal would preferably be contained in one to six capsules, tablets or beads. However, the present compositions may also be formulated in other convenient forms, such as an injectable solution or suspension, a spray solution or suspension, beads, a lotion, gum, lozenge, food or snack item. Food, snack, gum or lozenge items can include any ingestible ingredient, including sweeteners, flavorings, oils, starches, proteins, fruits or fruit extracts, vegetables or vegetable extracts, grains, animal fats or proteins. Thus, the present compositions can be formulated into cereals, snack items such as chips, bars, chewable candies or slowly dissolving lozenges.

The methods of the invention provide for modification of adipocyte physiology in a subject. While modification of adipocyte physiology to enhance lipogenesis or increase adiponectin secretion is desirable in and of itself, it is to be recognized that a modification of adipocyte physiology can have other salutary effects. The present formulation also reduces the inflammatory response and thereby promotes healing of, or prevents further damage to, the affected tissue.

According to the present invention, the animal may be a member selected from the group consisting of humans, non-human primates, dogs, cats, birds, horses, ruminants or other animals. In particular embodiments, the invention provides methods of treating of human beings. Administration of cinnamon or cinnamon extracts of the invention can be by any method available to the skilled artisan, for example, by oral, topical, transdermal, transmucosal, or parenteral routes. In addition, those of ordinary skill in the art can readily determine appropriate dosages that are necessary to achieve the desired therapeutic or prophylactic effect upon oral, parenteral, rectal and other administration forms. Typically, in vivo models (i.e., laboratory mammals) are used to determine the appropriate plasma concentrations necessary to achieve a desired mitigation of inflammation related conditions.

The following illustrative examples are provided to further elucidate, but not to limit, the invention.

Of the compounds and mixtures tested, however, combinations of cinnamon or cinnamon extract with metformin, glipizide or pioglitazone surprisingly possess synergistic lipogenic activity as compared to metformin, glipizide or pioglitazone alone. Thus, combinations of cinnamon or cinnamon extract with metformin, glipizide or pioglitazone represent an unexpected improvement in the art of increasing insulin sensitivity.

### EXAMPLE 1

### Synergistic Increases in Lipogenesis in Adipocytes by Combinations of Cinnamon Extract and Ground Cinnamon with Metformin

The Model—The 3T3-L1 murine fibroblast model is used to study the potential effects of compounds on adipocyte differentiation and adipogenesis. This cell line allows investigation of stimuli and mechanisms that regulate preadipocytes replication separately from those that regulate differentiation to adipocytes (Fasshauer, M, Klein, J, Neumann, S., Eszlinger, M, and Paschke, R. Hormonal regulation of adiponectin gene expression in 3T3-L1 adipocytes. Biochem Biophys Res Commun, 290: 1084-1089, 2002; Li, Y. and Lazar, M A. Differential gene regulation by PPARgamma agonist and constitutively active PPARgamma2. Mol Endocrinol, 16: 1040-1048, 2002) as well as insulin-sensitizing and triglyceride-lowering ability of the test agent (Raz, I., Eldor, R., Cernea, S., and Shafrir, E. Diabetes: insulin resistance and derangements in lipid metabolism. Cure through intervention in fat transport and storage. Diabetes Metab Res Rev, 2004).

As preadipocytes, 3T3-L1 cells have a fibroblastic appearance. They replicate in culture until they form a confluent monolayer, after which cell-cell contact triggers Go/G1 growth arrest. Terminal differentiation of 3T3-L1 cells to adipocytes depends on proliferation of both pre- and postconfluent preadipocytes. Subsequent stimulation with 3-isobutyl-1-methylxanthane, dexamethasone, and high dose of insulin (MDI) for two days prompts these cells to undergo postconfluent mitotic clonal expansion, exit the cell cycle, and begin to express adipocyte-specific genes. Approximately five days after induction of differentiation, more than 90% of the cells display the characteristic lipid-filled adipocyte phenotype. Assessing triglyceride synthesis of 3T3-L1 cells provides a validated model of the insulin-sensitizing and triglyceride-lowering ability of the test agent (Raz, I, Eldor, R., Cernea, S., and Shafrir, E. Diabetes: insulin resistance and derangements in lipid metabolism. Cure through intervention in fat transport and storage. Diabetes Metab Res Rev, 2004).

Test Materials—Metformin and the positive control troglitazone were obtained from Sigma, (St. Louis, Mo.). Sample A was Cinnulin PF™ containing Cinnamon extract (20:1) (Cinnamomi cassia) (bark,) 125 mg and vitamin C (ascorbyl palmitate) 5 mg. Other ingredients were hypo-allergenic plant fiber, and vegetable capsule. Sample B was 100% ground cinnamon bark obtained from Viable Herbal Solutions (Morrisville, Pa.). All standard reagents, unless otherwise indicted were obtained from Sigma. Testing was performed three times and combinations were assayed twice. The results presented are representative of this testing.

Cell culture and Treatment—The murine fibroblast cell line 3T3-L1 was obtained from the American Type Culture Collection (Manasus, Va.) and sub-cultured according to instructions from the supplier. For experiments, cells were cultured in DMEM containing 10% FBS-HI, with added 50 units penicillin/mL and 50 μg streptomycin/mL, and maintained in log phase prior to experimental setup. Cells were grown in a 5% CO₂ humidified incubator at 37° C. Components of the pre-confluent medium included (1) 10% FBS/DMEM containing 4.5 g glucose/L; (2) 50 U/mL penicillin; and (3) 50 μg/mL streptomycin. Growth medium was made by adding 50 mL of heat inactivated FBS and 5 mL of penicillin/streptomycin to 500 mL DMEM. This medium was stored at 4° C. Before use, the medium was warmed to 37° C. in a water bath.

3T3-T1 cells were seeded at an initial density of about 4×10⁴ cells/cm² in 24-well plates. For two days, the cells were allowed to grow to reach confluence. Following confluence, the cells were forced to differentiate into adipocytes by the addition of differentiation medium; this medium consisted of (1) 10% FBS/DMEM (high glucose); (2) 0.5 mM methylisobutylxanthine; (3) 0.5 μM dexamethasone and (4) 10 Hg/mL insulin. After three days, the medium was changed to post-differentiation medium consisting of 10 μg/mL insulin in 10% FBS/DMEM.

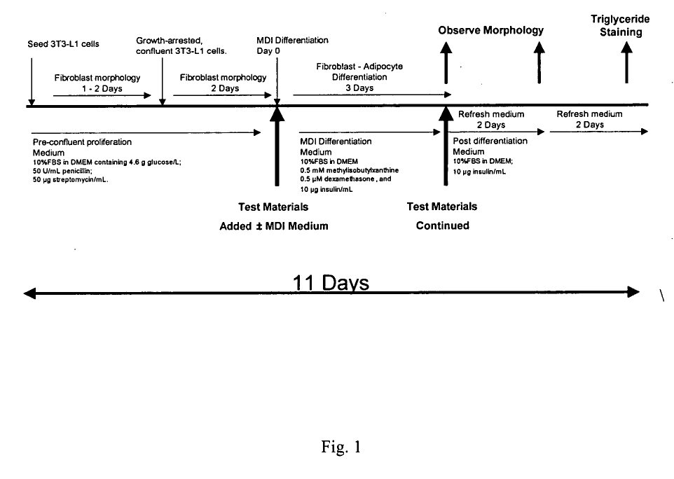

Test material was added in dimethyl sulfoxide at Day 0 of differentiation and every two days throughout the maturation phase (Day 7). Whenever fresh media was added, fresh test material was also added. As a positive control, troglitazone was added to achieve a final concentration of 4.4 μg/mL (10 μM). Metformin, Sample A and the metformin/Sample A combinations of 1:10, 1:5 and 1:2.5 were all tested at 50 μg test material/mL. Metformin, Sample B and the metformin/Sample B combination of 1:10 were all tested at 50 μg test material/mL. The complete procedure for differentiation and treatment of cells with test materials is outlined schematically in FIG. 1.

Oil Red O Staining—Differentiated 3T3-L1 cells were stained with Oil Red O according to the method of Kasturi and JoshI [Kasturi, R. and Joshi, V. C. Hormonal regulation of stearoyl coenzyme A desaturase activity and lipogenesis during adipose conversion of 3T3-L1 cells. J Biol Chem, 257: 12224-12230, 1982]. Monolayer cells were washed with PBS and fixed with 3.7% formaldehyde for ten minutes. Fixed cells were stained with 0.2% Oil Red O/isopropanol for one hour and the excess of stain was washed using a solution of 70% ethanol and water. The resulting stained oil droplets were dissolved with isopropanol and quantified by spectrophotometric analysis at 530 nm. Results were represented as a relative percentage of fully differentiated cells in the solvent controls.

Calculations—An estimate of the expected lipogenic effect of the metformin/Sample A or B composite was made using the relationship: [1/ED]=[X/ED]+[Y/ED], where X and Y were the relative fractions of each component in the test mixture and X+Y=1. An increase in adipogenesis of five percent or more from the expected value was considered synergistic. The value of five percent was chosen as repeated observations on the solvent control indicated that the upper 95% confidence limit was at 3.9 percent of the mean or 1.039.

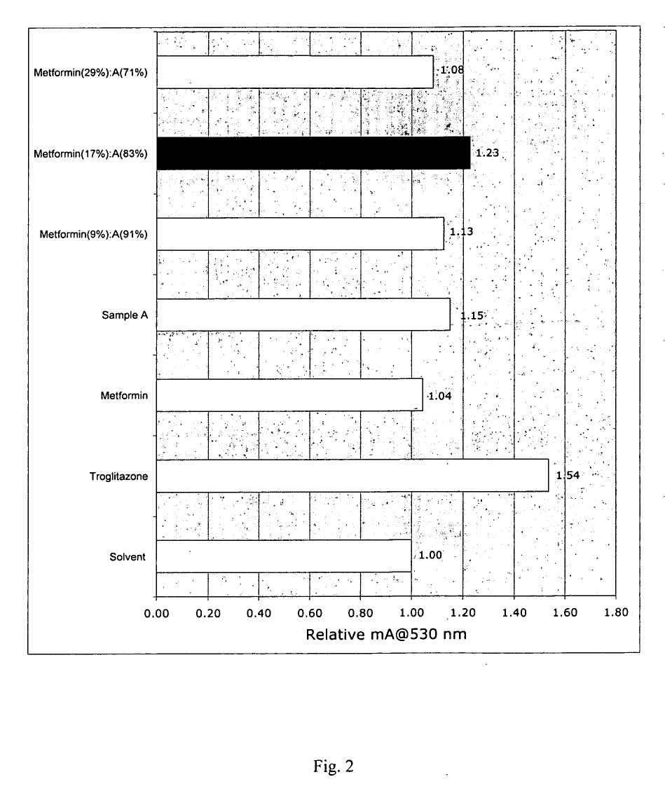

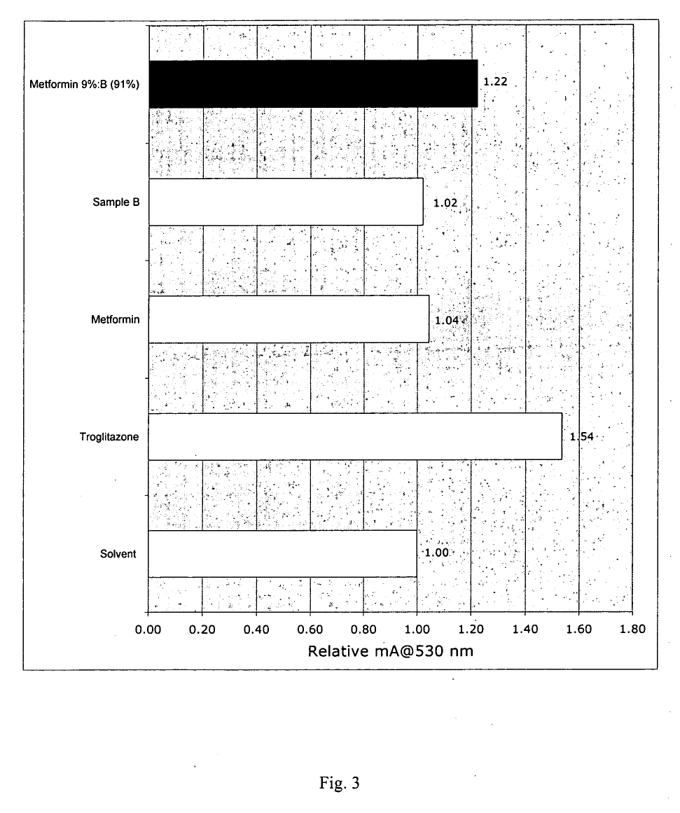

Results—Troglitazone, the positive control was highly lipogenic, increasing triglyceride content of the 3T3-L1 cells by 54 percent (FIG. 2). Sample A, the cinnamon extract was next in lipogenic activity with a 15 percent increase, while metformin demonstrated the lowest lipogenic increase at four percent (p<0.05). The combinations of metformin and Sample A were all lipogenic relative to the solvent control. Unexpectedly, the metformin/Sample A 1:5 combination demonstrated greater than expected lipogenic activity of 23 percent versus an expected 13 percent. Sample B, the ground cinnamon, was not lipogenic. With a 22 percent increase in triglyceride content, the nine percent metformin and 91 percent Sample B combination was, unexpectedly, highly lipogenic relative to the solvent control (FIG. 3).

Conclusions—Metformin and cinnamon extract combinations containing 10 to 28% metformin increased adipogenesis synergistically in the 3T3-L1 adipocyte model. The metformin and ground cinnamon combination containing nine percent metformin increased adipogenesis synergistically in the 3T3-L1 adipocyte model. As plain, ground cinnamon was not lipogenic, combinations of metformin and ground cinnamon containing more than nine percent metformin would also be expected to behave synergistically.

This example demonstrates that components of cinnamon that can be found in plain, ground cinnamon or the tested cinnamon extract synergistically enhanced the lipogenic effect of the diabetic drug metformin.

### EXAMPLE 2

### Synergistic Increases in Lipogenesis in Adipocytes by Combinations of Cinnamon Extract and Ground Cinnamon with Glipizide

The Model, Cell Culture, Oil Red O staining and Calculations were as described in Example 1.

Test materials—Glipizide was obtained from Sigma (St. Louis, Mo.) and Samples A and B and all reagents were as described in Example 1.

Test materials were added in dimethyl sulfoxide at Day 0 of differentiation and every two days throughout the maturation phase (Day 7). Whenever fresh media was added, fresh test material was also added. As a positive control, troglitazone was added to achieve a final concentration of 4.4 Hg/mL (10 μM). Glipizide, Sample A and the glipizide/Sample A combination of 1:10 were all tested at 50 μg test material/mL. Glipizide, Sample B and the glipizide/Sample B combinations of 1:10, 1:5 and 1:2.5 were all tested at 50 μg test material/mL. The complete procedure for differentiation and treatment of cells with test materials is outlined schematically in FIG. 1.

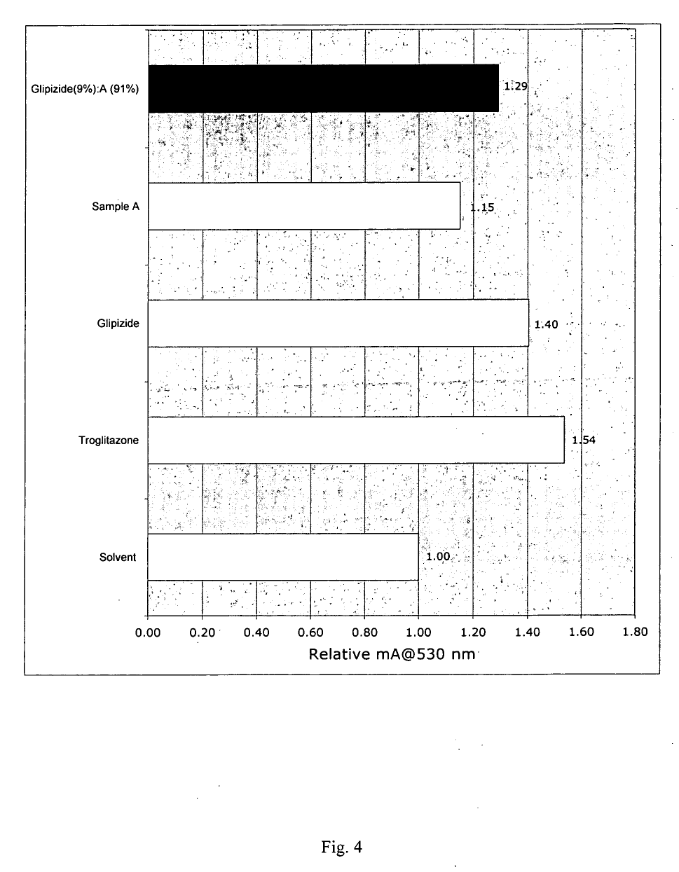

Results—Troglitazone, the positive control was highly lipogenic, increasing triglyceride content of the 3T3-L1 cells by 54 percent (FIG. 4). Glipizide was next in adipogenic activity with a 40 percent increase, while Sample A demonstrated the lowest response with a 15 percent adipogenic increase. With a 29 percent increase in triglyceride content, the nine percent glipizide and 91 percent Sample A combination was, unexpectedly, highly adipogenic relative to the solvent control. The expected increase in 3T3-L1 triglyceride content of the 1:10 glipizide:Sample A combination was 17 percent.

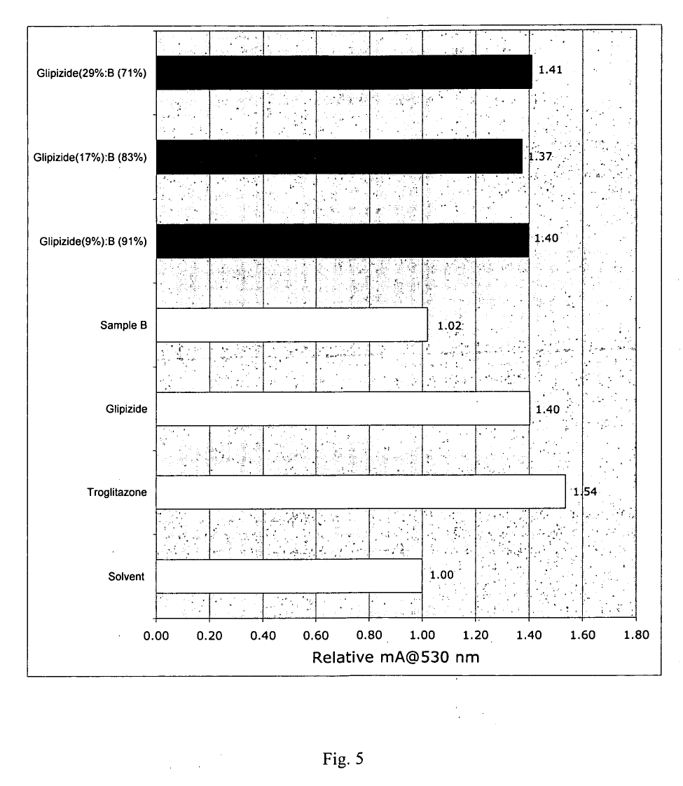

While Sample B was not lipogenic, glipizide and all combinations of glipizide with Sample B increased triglyceride biosynthesis in the 3T3-L1 cells relative to the solvent controls (FIG. 5). The 1:10, 1:5 and 1:2.5 combinations of glipizide with Sample B were expected to exhibit relative increases in triglyceride content, respectively, of 5, 7 and 11 percent. These combinations did, however, increase adipogenesis 40, 37 and 41 percent relative to the solvent controls.

Conclusions—The glipizide and cinnamon extract combination containing nine percent glipizide increased lipogenesis synergistically in the 3T3-L1 adipocyte model. Additionally, The glipizide and ground cinnamon combinations containing nine to 29 percent glipizide increased adipogenesis synergistically in the 3T3-L1 adipocyte model.

### EXAMPLE 3

### Cinnamon Powder and Cinnamon Extract Act Synergistically with Combinations of Metformin and Glipizide to Increase Triglyceride Incorporation in Insulin Resistant 3T3-L1 Adipocytes

The Model—The 3T3-L1 murine fibroblast model as described in Example 1 is used in these experiments. All chemicals and procedures used are as described in Example 1. Individually, metformin, glipizide, cinnamon powder and cinnamon extract are tested at 50 μg/mL. Combinations of metformin, glipizide and cinnamon powder or cinnamon extract are tested, respectively, at ratios of [1:1:100], [1:1:10], [1:1:5], [1:1:1], [5:5:1], [10:10:1], and [100:100:1] at a total concentration of 50 μg test material/mL. Thus, a test combination of 1:1:100 would contain 0.49 μg metformin, 0.49 μg glipizide, and 49 μg cinnamon or cinnamon extract/mL.

Calculations—An estimate of the expected lipogenic effect of the metformin/glipizide/cinnamon combinations is made using the relationship as described in Example 1.

Results—Combinations of metformin/glipizide and the cinnamon samples from [1:1:100] to [10:10:1] demonstrate unexpectedly greater lipid incorporation in adipocytes than expected based upon their individual activity. Thus, these combinations demonstrate synergy with respect to triglyceride incorporation in the 3T3-L1 adipocyte model and would be expected to act synergistically to increase insulin sensitivity, glucose utilization and normalize serum lipids, cardiovascular risk factors and markers of inflammation in animals. Additionally, such combinations would be expected to be useful to extend the range of positive benefits of diabetic polytherapy such as an increase in the number of patients responding.

### EXAMPLE 4

### Cinnamon Powder and Cinnamon Extract Act Synergistically with Pioglitazone to Increase Triglyceride Incorporation in Insulin Resistant 3T3-L1 Adipocytes

The Model—The 3T3-L1 murine fibroblast model as described in Example 1 is used in these experiments. All chemicals and procedures used are as described in Example 1. Pioglitazone is obtained as 45 mg pioglitazone tables from a commercial source as Actos® (Takeda Phamaceuticals, Lincolnshire, Ill.). The tablets are ground to a fine powder and tested at 5.0, 2.5, 1.25 and 0.635 μg pioglitazone/mL. Cinnamon powder and cinnamon extract are tested at 100, 50, 25 and 12 μg/mL. Combinations of pioglitazone and the two cinnamon samples are assayed, respectively, at ratios of [1:100], [1:50], [1:10], [1:1], [10:1], [50:1] and [100:1].

Calculations—An estimate of the expected lipogenic effect of the pioglitazone/cinnamon combinations is made using the relationship as described in Example 1.

Results—Pioglitazone as well as the cinnamon samples demonstrate dose-related increases in lipid incorporation in the insulin-resistant, 3T3-L1 adipocyte. Combinations of pioglitazone and the cinnamon samples from 1:50 to 50:1 demonstrate unexpectedly greater lipid incorporation in adipocytes than expected based upon their individual activity. Thus, these combinations demonstrate synergy with respect to triglyceride incorporation in the 3T3-L1 adipocyte model and would be expected to act synergistically to increase insulin sensitivity, glucose utilization and normalize serum lipids, cardiovascular risk factors and markers of inflammation in animals. Additionally, such combinations would be expected to be useful to increase the range of positive benefits of pioglitazone therapy such an increase in the number of patients responding to drugs of the thiazolidinedione class.

### EXAMPLE 5

### Normalization of Fasting Plasma Glucose and Insulin with a Coincident Decrease in Serum Triglycerides and Markers of Inflammation

A representative composition of the preferred embodiments as a medicant for the normalization of fasting plasma glucose and insulin as well as normalization of dyslipidemia would be in an oral formulation, i.e. tablets or gel caps that would supply one of the following combinations: 0.1 to 10 mg ground cinnamon/kg per day and an effective amount of metformin; 0.01 to 10 mg cinnamon extract/kg per day and an effective amount of glipizide for a 70 kg person.

Normalization of fasting plasma glucose and insulin would be expected to occur following ten to 20 doses. Furthermore, increases in serum high density lipoprotein (HDL) of greater than 20 percent and decreases of serum triglycerides of greater than 35 percent would be likely observed. This result would be expected in all animals.

All publications and patent applications mentioned in this specification are indicative of the level of skill of those skilled in the art to which this invention pertains. All publications and patent applications are herein incorporated by reference to the same extent as if each individual publication or patent application was specifically and individually indicated to be incorporated by reference.

The invention now having been fully described, it will be apparent to one of ordinary skill in the art that many changes and modifications can be made thereto without departing from the spirit or scope of the appended claims.

We claim:

1. A composition comprising therapeutically effective amounts of cinnamon and a hypoglycemic therapeutic selected from the group consisting of a biguanide, a sulfonylurea, a thiazolidinedione and mixtures thereof, said composition providing a synergistic lipogenic activity when administered to a warm blooded animal.

2. The composition according to claim 1, wherein said biguanide is a member selected from the group consisting of merformin, chloroguanide, phenformin, and buformin.

3. The composition according to claim 1, wherein said sulfonylurea is a member selected from the group consisting of glipizide, tolbutamide, chlorpropamide, tolazamide, acetohexamide, glyburide, and gliclazide.

4. The composition according to claim 1, wherein said thiazolidinedione is a member selected from the group consisting of rosiglitazone, pioglitazone and troglitazone.

5. The composition according to claim 1, wherein said cinnamon is in form of powder or extract.

6. The composition of claim 5 wherein the cinnamon extract is made by extracting a cinnamon bark powder with water, steam, superheated water, glycerin, ethylene glycol, methanol, diethylene glycol, ethanol, acetic acid, 1-propoanol, 1-butanol, acetonitrile, dimethyl sulfoxide, dimethyl formamide, t-butyl alcohol, acetone, 2-butanone, methylene chloride, chloroform, diglyme, dimethyoxy ethane, ethyl acetate, thetrahydrofuran, dioxane, methyl t-butyl ether, ether, benzene, toluene, p-xylene, carbon tetrachloride, heptane, hexane, pentane, octanol, cyclohexane, supercritical CO₂, liquid CO₂, liquid N₂ or any combinations of such materials.

7. A method for the treatment or reducing the symptoms of metabolic disorders or inflammatory conditions in a warm blooded animal comprising administering to the warm blooded animal a composition comprising cinnamon and a hypoglycemic therapeutic selected from the group consisting of a biguanide, a sulfonylurea, a thiazolidinedione and mixtures thereof in a therapeutically effective amount to provide a synergistic lipogenic activity to the warm blooded animal.

8. The method according to claim 7, wherein said metabolic disorder is a member selected from the group consisting of type 2 diabetes, diabetic complications, insulin sensitivity, hyperglycemia, dyslipidemia, insulin resistance, metabolic syndrome, obesity or body weight gain in the warm blooded animal.

9. The method according to claim 7, wherein said biguanide is a member selected from the group consisting of merformin, chloroguanide, phenformin, and buformin.

10. The method according to claim 7, wherein said sulfonylurea is a member selected from the group consisting of glipizide, tolbutamide, chlorpropamide, tolazamide, acetohexamide, glyburide, and gliclazide.

11. The method according to claim 7, wherein said thiazolidinedione is a member selected from the group consisting of rosiglitazone, pioglitazone and troglitazone.

12. The method according to claim 7, wherein said cinnamon is in form of powder or extract.

13. The method according to claim 12 wherein the cinnamon extract is made by extracting a cinnamon bark powder with water, steam, superheated water, glycerin, ethylene glycol, methanol, diethylene glycol, ethanol, acetic acid, 1-propoanol, 1-butanol, acetonitrile, dimethyl sulfoxide, dimethyl formamide, t-butyl alcohol, acetone, 2-butanone, methylene chloride, chloroform, diglyme, dimethyoxy ethane, ethyl acetate, thetrahydrofuran, dioxane, methyl t-butyl ether, ether, benzene, toluene, p-xylene, carbon tetrachloride, heptane, hexane, pentane, octanol, cyclohexane, supercritical CO₂, liquid CO₂, liquid N₂ or any combinations of such materials.

14. A kit for use in the treatment or reducing the symptoms of metabolic disorders or inflammatory conditions in a warm blooded animal comprising: an effective amount of cinnamon, an effective amount of a hypoglycemic therapeutic selected from the group consisting of a biguanide, a sulfonylurea, a thiazolidinedione and mixtures thereof, and instructions describing a method of administering said cinnamon and hypoglycemic therapeutic to provide a synergistic lipogenic effect in said warm blooded animal.

15. The kit according to claim 14, wherein said metabolic disorder is a member selected from the group consisting of type 2 diabetes, diabetic complications, insulin sensitivity, hyperglycemia, dyslipidemia, insulin resistance, metabolic syndrome, obesity or body weight gain in the warm blooded animal.

16. The kit according to claim 14, wherein said biguanide is a member selected from the group consisting of merformin, chloroguanide, phenformin, and buformin.

17. The kit according to claim 14, wherein said sulfonylurea is a member selected from the group consisting of glipizide, tolbutamide, chlorpropamide, tolazamide, acetohexamide, glyburide, and gliclazide.

18. The kit according to claim 14, wherein said thiazolidinedione is a member selected from the group consisting of rosiglitazone, pioglitazone and troglitazone.

19. The kit according to claim 14, wherein said cinnamon is in form of powder or extract.

20. The kit according to claim 19, wherein the cinnamon extract is made by extracting a cinnamon bark powder with water, steam, superheated water, glycerin, ethylene glycol, methanol, diethylene glycol, ethanol, acetic acid, 1-propoanol, 1-butanol, acetonitrile, dimethyl sulfoxide, dimethyl formamide, t-butyl alcohol, acetone, 2-butanone, methylene chloride, chloroform, diglyme, dimethyoxy ethane, ethyl acetate, thetrahydrofuran, dioxane, methyl t-butyl ether, ether, benzene, toluene, p-xylene, carbon tetrachloride, heptane, hexane, pentane, octanol, cyclohexane, supercritical CO₂, liquid CO₂, liquid N₂ or any combinations of such materials.
